(19) United States
(12) Patent Application Publication
(10) Pub. No.: US 2006/0241480 A1
(43) Pub. Date: Oct. 26, 2006

(54) Endoscopic medical method and associated device

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/386,504

(22) Filed: Mar. 22, 2006

Related U.S. Application Data
(60) Provisional application No. 60/670,325, filed on Apr. 12, 2005.

(51) Int. Cl. A61B 8/14

(52) U.S. Cl. 600/466

(57) ABSTRACT

A surgical method includes inserting a distal end portion of an endoscope into a patient along a given path and inserting a distal end portion of a flexible ultrasonic probe device into the patient along the given path. The ultrasound probe device is operated to generate an image of organic tissue structures internal to the patient. Subsequently, a surgical operation is conducted on the organic tissue structures.

### CROSS-REFERENCE TO RELATED APPLICATION

This application claims the benefit of U.S. Provisional Patent Application No. 60/670,325 filed Apr. 12, 2005.

### BACKGROUND OF THE INVENTION

This invention relates to minimally invasive medical procedures of a kind carried out with an endoscope. This invention also relates to a device useful in such procedures.

One kind of endoscopic procedure is described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. These procedure can be called “trans-organ procedures.”

### OBJECTS OF THE INVENTION

It is an object of the present invention to provide an improvement for use in endoscopic procedures utilizing a flexible endoscope insertion member.

It is another object of the present invention to provide a method and/or an associated device useful in trans-organ endoscopic procedures.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A medical, surgical and/or diagnostic, device in accordance with the present invention includes a flexible elongate shaft, a first electroacoustic transducer element disposed at a distal end of the elongate shaft for converting electrical energization waveform into an ultrasonic pressure wave, and a second electroacoustic transducer element disposed at a distal end of the elongate shaft for converting incoming ultrasonic pressure waves into electrical signals.

The shaft may have a diameter sufficiently small to insert the shaft through a working channel of an endoscope assembly. Thus, the shaft and the operating tip with the transducer elements may be inserted into a patient along a curvilinear path having a plurality of turns or bends.

For instance, where the endoscope is encased wholly or partially in a sheath, the flexible shaft of the surgical device may be inserted through a tube attached to the sheath and defining the working channel.

The device may further include image processing electronic componentry operatively connected to the second electroacoustic transducer element for generating an image of organic tissues proximate the distal end of the elongate shaft.

A surgical method in accordance with the present invention includes inserting a distal end portion of an endoscope into a patient along a given path, inserting a distal end portion of an ultrasonic probe device into the patient along the given path, operating the ultrasound probe device to generate an image of organic tissue structures internal to the patient, and subsequently conducting a surgical operation on the organic tissue structures.

Where the endoscope is provided with a sheath having a longitudinally extending channel and the ultrasonic probe device has an elongate shaft, the method may further comprise inserting the shaft through the channel prior to the operating of the ultrasound probe device to generate the image.

The method of the invention is especially useful where organic tissue structures are hidden from detection via optics of the endoscope. The ultrasonic probe device is operated to obtain an image of the hidden organic tissue structures.

The distal end portion of the endoscope and the distal end portion of the ultrasound probe may be inserted into the patient via a natural body opening of the patient, pursuant to a trans-organ surgical procedure as described in U.S. Pat. Nos. 5,297,536 and 5,458,131. The distal end portion of the endoscope and the distal end portion of the ultrasound probe are further inserted through a wall of an internal hollow organ of the patient into an internal body cavity of the patient, the organic tissue structures being located in the internal body cavity.

The internal tissue structures may include a common bile duct and cystic duct.

### BRIEF DESCRIPTION OF THE DRAWING

FIG. 1 is a schematic partial longitudinal cross-section of a human patient, showing an abdomen-insufflation step in a trans-organ surgical procedure.

FIG. 2 is a schematic cross-sectional view of a hollow internal organ of a patient, showing a step in a modified abdomen-insufflation step of a trans-organ surgical procedure.

FIG. 3A is a schematic partial cross-sectional view of the organ of FIG. 2, showing a step in the deployment of a trans-organ port.

FIG. 3B is a schematic partial cross-sectional view similar to FIG. 3A, showing a subsequent step in the deployment of the trans-organ port of FIG. 3A.

FIG. 4 is a schematic perspective view of an endoscopic surgical assembly in accordance with the present invention, showing a step in the use of the assembly in a trans-organ surgical procedure involving a common bile duct and cystic duct of a patient.

FIG. 5 is a schematic perspective view of a distal end or head portion of an ultrasonic medical instrument included in the assembly of FIG. 5.

### DETAILED DESCRIPTION

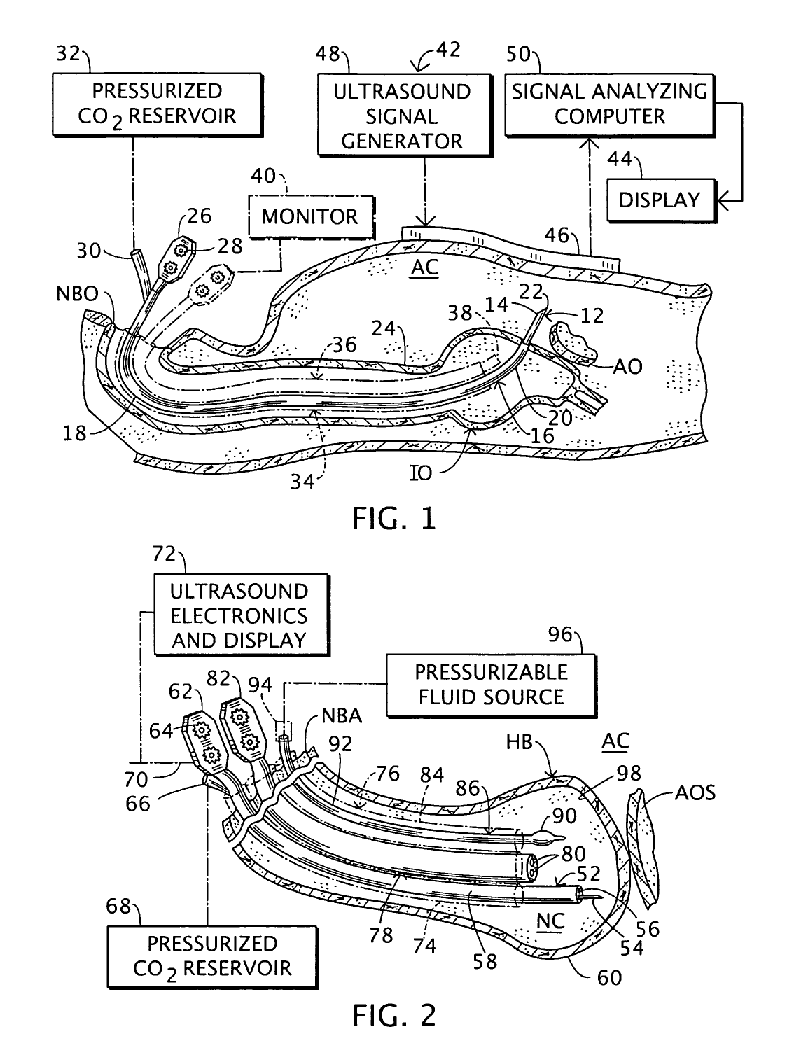

As illustrated in FIG. 1, a method for insufflating a patient's abdominal cavity AC during a trans-organ surgical procedure as described in U.S. Pat. Nos. 5,297,536 and 5,458,131 (both incorporated by reference herein) includes inserting a distal end portion of an insufflation instrument 12 into a hollow internal organ IO of the patient via a natural body opening NBO of the patient. Internal organ IO, which defines an internal body cavity IC, may be the stomach, the urinary bladder, the colon, or the vagina, while the natural body opening NBO is the mouth, the urethral orifice, the anus, or the vaginal orifice.

Instrument 12 includes a hollow needle 14 at a distal end of a tubular flexible shaft member 16. The inserting of needle 14 into the patient may include passing the needle and a distal end portion of the shaft member 16 along a nonlinear path (not separately designated) having at least one bend or turn 18, 20. A distal or free end 22 of needle 14 is inserted through a wall 24 of the organ IO and thereafter a pressurized fluid is conveyed through shaft 16 and needle 14 into the patient's abdominal cavity AC on the side of wall 24 opposite the internal body cavity IC.

Shaft 16 may be coupled at a proximal end to a handpiece 26 provided with one or more steering knobs 28 and a port 30 connected to a source 32 of pressurized carbon dioxide gas.

Needle 14 and the distal end portion of shaft 16 may be inserted into the patient and particularly into organ IO through a tubular working channel 34 of an endoscope sheath 36. Needle 14 may be disposed inside channel 34 at the time of manufacture and thus inserted into the patient together with an endoscope 38. Endoscope 38 is connected to a video monitor 40 for enabling a surgeon to view the insertion path and the inner surface of wall 24 during the perforation or penetration of the organ wall by needle 14.

If needle 14 is inserted into cavity IC together with endoscope 38 and sheath 36, channel 34 may be formed with an inner lining that is made of a hard, puncture resistant material. Alternatively, needle 14 may be housed in a dedicated deployment tube (not illustrated) that may be inserted into channel 34 after the insertion of endoscope 38 and sheath 36 into the patient.

As an alternative or additional method for the monitoring of organ IO and needle 14 prior to and during the penetration of wall 24 by the needle, a wireless scanning apparatus such as an ultrasound scanner 42 may be used to view internal organ IO and other structures inside the patient on a display or monitor 44. Ultrasound scanner 42 may particularly include a transducer carrier 46 placed in contact with the patient, an ultrasonic waveform generator 48 operatively connected to the carrier for energizing the transducers (e.g., piezoelectric crystals, not shown) thereof, and a computer 50 functioning as an ultrasound signal analyzer operatively connected to the carrier for receiving therefrom signals encoding ultrasonic waves reflected from internal structures.

An entirely electronic (no moving parts) ultrasound scanner suitable for the present purposes is disclosed in the following patents: U.S. Pat. No. 5,871,446, U.S. Pat. No. 6,023,032, U.S. Pat. No. 6,319,201, U.S. Pat. No. 6,106,463, and U.S. Pat. No. 6,306,090. Other kinds of ultrasound scanning devices, as well as magnetic resonance imaging, X-ray machines, and CAT scanners, may also be suitable for present purposes, i.e., for monitoring the shapes and relative positions of organ IO and other internal tissue structures.

Ultrasound scanner 42 is operated and display or monitor 44 viewed in order to determine whether a selected location along organ wall 24 is free and clear of other intra-abdominal organs or whether organ wall 24 at a selected location lies against another organ AO or the patient. This determination is made prior to the penetration of wall 24 by needle 14, to ensure that needle 14 does not enter another organ AO and conduct insufflation fluid into that other organ. Instead, the point of penetration of needle 14 through wall 24 is selected to avoid adjacent organ structures AO, so that needle 14 subsequently conducts carbon dioxide gas into abdominal cavity AC.

FIG. 2 depicts an alternative abdomen inflation method wherein the detection of an adjacent organ structure AOS inside the patient is accomplished via an ultrasound probe 52 that is inserted into a hollow body organ HB via a natural body opening or aperture NBA together with an insufflation needle 54. Probe 52 may specifically include one or more ultrasound transducers 56 disposed in the end of an elongate flexible tubular member 58 from which needle 54 is ejected into a natural body cavity NC defined by a wall 60 of organ HB. Needle 54 is coupled to the distal end of an elongate flexible tubular shaft (not shown) such as shaft 16 in FIG. 1. Such an elongate flexible tubular shaft is insertable through a lumen or channel inside tubular member 58. That lumen or channel may be lined at a distal end with a layer of a hard low-friction material such as polytetrafluoroethylene, to facilitate the ejection of needle 54.

Probe 52 may include a handpiece 62 connected to a proximal end of tubular member 58, the handpiece being provided with steering controls 64 and a port 66 for coupling to a source or reservoir 68 of pressurized carbon dioxide gas (possibly in liquid form). Handpiece 62 is also provided with a connector 70 for forming an electrically conductive link to an ultrasound electronics apparatus and display 72. This electrically conductive link enables the transmission of ultrasound pressure waves and the sensing of incoming reflected waveforms by 56 under the control of ultrasound electronics apparatus 72.

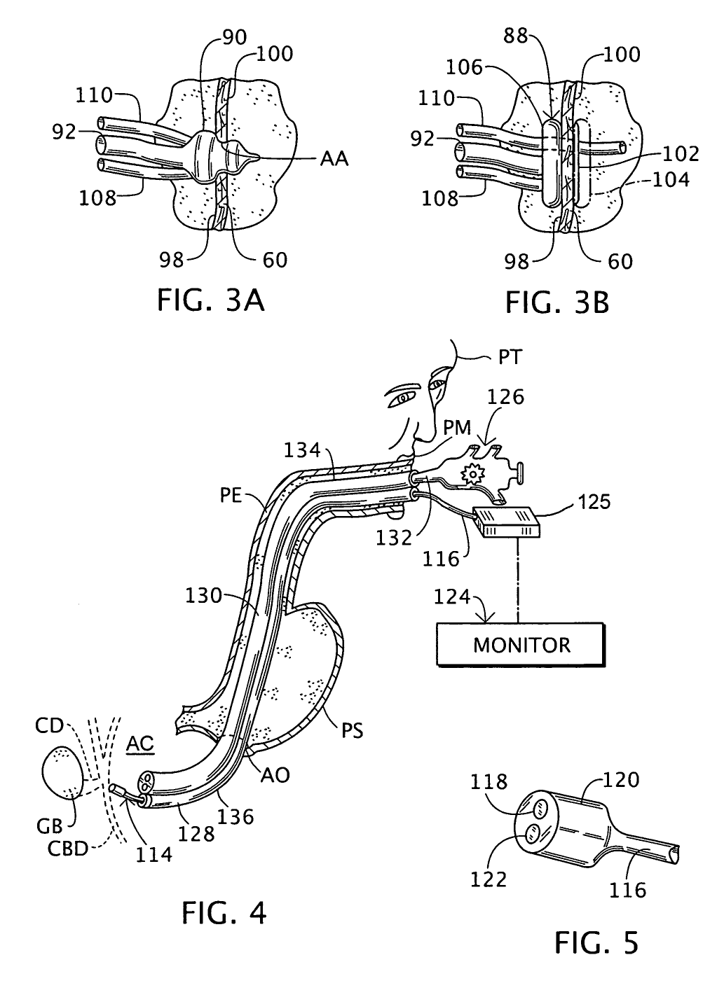

Probe 52 and needle 54 may be inserted into cavity NC through a collapsible tubular channel element 74 of an endoscope sheath 76 attached to and surrounding an endoscope 78. Endoscope 78 has optical elements 80 and a handpiece 82. Sheath 76 may be provided with a second tubular channel 84 through which an instrument 86 is inserted into cavity NC of organ HB for deploying a port element 88 (FIG. 3B) in organ wall 60. At the time of insertion, instrument 86 includes port element 88 in a collapsed or folded insertion configuration 90 and an elongate flexible tubular shaft 92. At a proximal end, shaft 92 includes a port or connector schematically represented at 94 for coupling the shaft to a source 96 of pressurized fluid such as saline solution.

Upon the insertion of needle 54 and probe 52 into cavity NC of organ HB and the subsequent placement of transducer elements 56 into contact with a proximal surface 98 (FIGS. 3A and 3B) of organ wall 60, ultrasound electronics 72 are operated to scan through the organ wall for the presence of an adjacent organ structure AOS in contact with or proximate to a distal surface 100 (FIGS. 3A and 3B) of wall 60. If an adjacent organ structure AOS is detected, probe 52 is manipulated from outside the patient to reposition the probe head (not separately enumerated) including transducer elements 56 at another location along proximal surface 108 of organ wall 60. Upon failing to detect an adjacent organ structure AOS alongside distal surface 100 of organ wall 60, the operating surgeon moves needle 54 in a distal direction to penetrate through wall 60. Upon completed penetration, carbon dioxide gas from source or reservoir 68 through the flexible shaft or tube (not shown) and into the abdominal cavity AC via needle 54.

Upon an insufflation of the abdominal cavity by this method, needle 54 is withdrawn from organ wall 60. Then instrument 86 in moved forward so that the collapsed form 90 of port element 88 may be pushed partially through organ wall 60 at the former site of needle penetration (see FIG. 3A). Subsequently, a disk 102 or balloon 104 on the distal side of port element 88 is expanded from the collapsed configuration 90 of the port element, as shown in FIG. 3B, while a balloon or bladder element 106 on the proximal side of the port element is inflated to an expanded configuration.

Disk 102 is made of a flexible sheet material. Disk 102 (or balloon 104) and balloon 106 define respective apertures (not shown) that are aligned with one another to define a hole for the passage of a medical instrument (not shown) through the port element 88. Balloon 106 is attached to disk 102 and has an inflation tube 108 for enabling an introduction of a pressurizing fluid into the balloon to expand the balloon from a collapsed insertion configuration to an inflated use configuration. (In the case of balloon 104 in place of disk 102, balloons 104 and 106 communicate with one another to enable an inflating of both balloons via saline or other fluid conveying through tube 108.)

At least one valve element in the form of a self-sealing membrane or film (not shown) may be provided on port element 88 for forming a seal about the shaft of a medical instrument inserted through the port element into abdominal cavity AC during a trans-organ procedure as described in U.S. Pat. Nos. 5,297,536 and 5,458,131. The valve element or self-sealing membrane may be realized as a resilient annular flange or film material about at least one of the apertures in the disk 102 and the balloon 106.

Another elongate tube 110 may be attached to port element 88, traversing the port element, for the introduction of gas (e.g., carbon dioxide) to maintain pneumoperitoneum in abdominal cavity AC during a trans-organ procedure as described in U.S. Pat. Nos. 5,297,536 and 5,458,131.

Disk 102 may be provided along an edge or periphery with a ring (not shown) of a resilient material stiffer than the flexible sheet material of the disk. The ring assists in spreading disk 102 during a deployment procedure, after a passing of disk 102 in a collapsed form through the artificial aperture AA formed in organ wall 60, for instance, by needle 54 or an incising instrument (not shown). Alternatively, where the ring is omitted, disk 102 is held in an opened configuration by the higher gas pressure in the abdominal cavity AC.

In a trans-organ surgical procedure as described in U.S. Pat. Nos. 5,297,536 and 5,458,131, port element 88 is connected to wall 60 and disposed in artificial aperture AA to keep that aperture open during a surgical procedure conducted via organ HB and natural body cavity NC, as described in U.S. Pat. Nos. 5,297,536 and 5,458,131. Upon completed deployment of port element 88, disk 102 (or balloon 104) and balloon 106 sandwich organ wall 60 and maintain access to abdominal cavity AC via aperture AA.

Port element 88 may be used upon completion of the insufflation operation discussed above with reference to FIG. 1. Needle 54 may be inserted into the patient along a nonlinear path having at least one bend or turn. Alternatively, in some cases, the needle 54 may be inserted into the patient along a linear path.

As illustrated in FIGS. 4 and 5, an ultrasonic diagnostic or surgical device 114 for use in flexible endoscopic surgery includes an elongate flexible shaft 116, a first electroacoustic transducer element 118 (FIG. 5) disposed at in an operating head or tip 120 at the distal end of the elongate shaft for converting an electrical energization waveform into an ultrasonic pressure wave, and a second electroacoustic transducer element 122 (FIG. 5) disposed in head 120 of the elongate shaft for converting incoming ultrasonic pressure waves into electrical signals. Transducers 118 and 122 are operatively connected to an electronic component 124 that includes circuitry and optionally programming for generating ultrasonic-frequency electrical signals energizing transducer 118 and for analyzing ultrasonic-frequency echo waveform sensed by transducer 122. Electronic component 124 further includes image processing electronic componentry operatively connected to transducer element 122 for generating an image of organic tissues proximate the distal end of elongate shaft 116.

Shaft 116 has a diameter sufficiently small to insert the shaft through a working channel of an endoscope assembly 126. In the embodiment of FIG. 4, the endoscope working channel is formed by a tube 128 extending along and integrally connected to a tubular sheath 130 that surrounds an endoscope insertion member 132. Shaft 116 and operating head 120 are inserted into a patient PT along a curvilinear path (not separately designated) having a plurality of turns or bends 134, 136.

FIG. 4 shows a step in an endoscopic trans-organ surgical or diagnostic procedure involving detection of a common bile duct CBD and cystic duct CD. A distal end portion of endoscope insertion member 132, encased in sheath 130, is inserted into patient PT along a path through the patient mouth PM (a natural body opening), the patient's esophagus PE and the patient's stomach PS. Pursuant to the techniques of U.S. Pat. Nos. 5,297,536 and 5,458,131, an artificial opening AO is formed in a wall of stomach PS. The distal tip of endoscope insertion member 132 is then passed through opening AO into the patient's abdominal cavity AC. Shaft 116 is pushed in the distal direction so that head 120 protrudes from working channel tube 128. The optics (not designated) of endoscope assembly 126 are used to visualize internal organs of the patient PT, for example, for purposes of removing the patient's gall bladder GB. The cystic duct CD and common bile duct CBD may be covered by connective and other tissues and therefore not possible to detect via the optics of endoscope assembly 126. In that case, ultrasound probe device 114 is used to ultrasonically locate the cystic duct CD and the common bile duct CBD. More specifically, device 114 is operated to generate an image on a monitor or display screen 124 of organic tissue structures internal to the patient PT. Subsequently, a trans-organ endoscopic cholecystectomy is performed wherein flexible surgical instruments are inserted through other working channels (not shown) of endoscope assembly 126.

Shaft 116 may be inserted through working channel tube 128 prior to the deployment of endoscope insertion member 132 in the patient. In that case, device 114 is inserted into the patient simultaneously with the endoscope insertion member 132. Alternatively, shaft 116 of device 114 may be inserted through an endoscope working channel (e.g., tube 128) after the endoscope has been used to visually inspect a prospective surgical site.

The surgical tools and instruments described hereinabove may be provided in various combinations as kits for facilitating not only the distribution of the surgical tools, instruments and closure elements but also the deployment and utilization of the surgical tools and uments in the operating room.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical device including:
an elongate flexible shaft;
a first electroacoustic transducer element disposed at a distal end of said elongate flexible shaft for converting an electrical energization waveform into an ultrasonic pressure wave; and
a second electroacoustic transducer element disposed at said distal end of said elongate flexible shaft for converting incoming ultrasonic pressure waves into electrical signals.

2. The device defined in claim 1 wherein said shaft has a diameter sufficiently small to insert said elongate flexible shaft through a working channel of an endoscope assembly.

3. The device defined in claim 2, further comprising an endoscope sheath including a tube defining said working channel.

4. The device defined in claim 1, further comprising image processing electronic componentry operatively connected to said second electroacoustic transducer element for generating an image of organic tissues proximate said distal end of said elongate flexible shaft.

5. The device defined in claim 1, further comprising a connector 70 at a proximal end of said elongate flexible shaft for forming an electrically conductive link to an ultrasound electronics apparatus.

6. The device defined in claim 1, further comprising:
a handpiece connected to a proximal end of said elongate flexible shaft; and
steering controls on said handpiece

7. A surgical method comprising:
inserting a distal end portion of an endoscope into a patient along a given path;
inserting a distal end portion of an ultrasonic probe device into the patient along said given path;
operating said ultrasound probe device to generate an image of organic tissue structures internal to the patient; and
subsequently conducting a surgical operation on said organic tissue structures.

8. The method defined in claim 7 wherein said endoscope is provided with a sheath having a longitudinally extending channel, said ultrasonic probe device having an elongate shaft, further comprising inserting said shaft through said channel prior to the operating of said ultrasound probe device to generate said image.

9. The method defined in claim 7 wherein organic tissue structures are hidden from detection via optics of said endoscope, said ultrasonic probe device being operated to obtain an image of the hidden organic tissue structures.

10. The method defined in claim 7 wherein the distal end portion of said endoscope and the distal end portion of said ultrasound probe are inserted into the patient via a natural body opening of the patient.

11. The method defined in claim 7 wherein the distal end portion of said endoscope and the distal end portion of said ultrasound probe are further inserted through a wall of an internal hollow organ of the patient into an internal body cavity of the patient, said organic tissue structures being located in said internal body cavity.

12. The method defined in claim 7 wherein said internal tissue structures include a common bile duct and cystic duct.
